(19) United States
(12) Patent Application Publication
(10) Pub. No.: US 2019/0316102 A1
(43) Pub. Date: Oct. 17, 2019

(54) MODIFICATION OF IMMUNE-RELATED GENOMIC LOCI USING PAIRED CRISPR NICKASE RIBONUCLEOPROTEINS

(71) Applicant: SIGMA-ALDRICH CO. LLC, St. Louis, MO (US)

(72) Inventors: Qingzhou Ji, St. Louis, MO (US); Gregory D. Davis, St. Louis, MO (US); Jacob T. Lamberth, St. Louis, MO (US)

(73) Assignee: SIGMA-ALDRICH CO. LLC, St. Louis, MO (US)

(21) Appl. No.: 16/383,135

(22) Filed: Apr. 12, 2019

Related U.S. Application Data
(60) Provisional application No. 62/657,488, filed on Apr. 13, 2018.

(51) Int. Cl. C12N 9/22; A61K 35/17; C12N 15/90; C12N 15/11

(52) U.S. Cl. CPC C12N 9/22; A61K 35/17; C12N 2800/80; C12N 15/11; C12N 2310/20; C12N 15/907

(57) ABSTRACT

Paired CRISPR nickase ribonucleoproteins engineered to target immune-related genomic loci and methods of using said ribonucleoproteins to modify the immune-related genomic loci.

### CROSS-REFERENCE TO RELATED APPLICATIONS

This application claims priority to U.S. Provisional Application Ser. No. 62/657,488, filed Apr. 13, 2018, the disclosure of which is hereby incorporated by reference in its entirety.

### SEQUENCE LISTING

This application contains a Sequence Listing that has been submitted in ASCII format via EFS-Web and is hereby incorporated by reference in its entirety. The ASCII copy, created on Apr. 12, 2019, is named P18_061 US_SL.txt, and is 18,220 bytes in size.

### FIELD

The present disclosure relates to paired CRISPR nickase ribonucleoproteins engineered to target immune-related genomic loci and methods of using to modify the immune-related genomic loci.

### BACKGROUND

Immunotherapy is a powerful treatment option that harnesses the immune system to fight cancer, infection, and other diseases. Traditional immunotherapy comprises the use of substances such as vaccines, monoclonal antibodies, cytokines, etc. to stimulate or suppress the immune system and other compounds. In recent years, genome editing is being used to modify the DNA of cells to engineer better functioning cells for use in immunotherapy. Zinc finger nucleases and CRISPR nucleases are being used to engineer disease fighting cells. However, these genome targeting techniques are hindered by low targeting frequencies and off-target effects. Thus, there is a need for improved and more precise genome editing at immune-related genomic loci.

### SUMMARY OF THE DISCLOSURE

Among the various aspects of the present disclosure is the provision of a method for modifying an immune-related genomic locus in a eukaryotic cell. The method comprises introducing into the eukaryotic cell Clustered Regularly Interspaced Short Palindromic Repeats (CRISPR) nickase ribonucleoproteins (RNPs) comprising a pair of guide RNAs designed to hybridize with target sequences in the immune-related genomic locus, such that repair of a double-stranded break created by the CRISPR nickase RNPs results in modification of the immune-related genomic locus.

Another aspect of the disclosure is directed to a composition comprising a CRISPR nickase and a pair of guide RNAs engineered to target an immune-related genomic locus.

Another aspect of the disclosure is directed to a method of treating cancer in a subject. The method comprises modifying an immune-related genomic locus in an ex vivo eukaryotic cell in accordance with the methods described herein to prepare a modified eukaryotic cell, and delivering to the subject the modified eukaryotic cell.

Other objects and features will be in part apparent and in part pointed out hereafter.

### DETAILED DESCRIPTION

The present disclosure provides paired CRISPR nickase ribonucleoproteins engineered to target immune-related genomic loci, and methods of using said paired CRISPR nickase RNPs to modify the immune-related loci. The compositions and methods disclosed herein can be used for targeted immunotherapy, e.g., cancer immunotherapy.

### (I) CRISPR Nickase Ribonucleoproteins

One aspect of the present disclosure provides paired CRISPR nickase ribonucleoproteins (RNPs) targeted to genomic loci involved in immune function. Paired CRISPR nickase RNPs comprise at least one pair of offset guide RNAs designed to hybridize with target sites in the genomic locus of interest such that the coordinated nicking of the nickases results in a double-stranded break in the genomic locus, which when repaired by a cellular DNA repair process results in a modification to the genomic locus.

### (a) Target Genomic Loci

In general, the paired CRISPR nickase RNPs can be engineered to target an immune-related genomic locus. The genomic loci may, for example, correlate with the loss of effector function of the immune cells and are advantageously distinct, separate or uncoupled from, or independent of the immune cell activation status. Alternatively, the genomic loci may, for example, correlate with immune cell activation and are advantageously distinct, separate or uncoupled from, or independent of the immune cell dysfunction status. Thus, in various embodiments, for example, dysfunctional loci may be targeted while leaving activation loci intact.

In other embodiments, the paired CRISPR nickase RNPs can be engineered to target to a genomic locus chosen from 2B4 (CD244), 4-1BB (CD137), A2aR, AAVS1, ACTB, ALB, B2M, B7.1, B7.2, B7-H2, B7-H3, B7-H4, B7-H6, BAFFR, BCL11A, BLAME (SLAMF8), BTLA, butyrophilins, CCR5, CD100 (SEMA4D), CD103, CD11a, CD11b, CD11c, CD11d, CD150, IPO-3), CD160, CD160 (BY55), CD18, CD19, CD2, CD27, CD28, CD29, CD30, CD4, CD40, CD47, CD48, CD49a, CD49D, CD49f, CD52, CD69, CD7, CD83, CD84, CD8alpha, CD8beta, CD96 (Tactile), CDS, CEACAM1, CRTAM, CTLA4, CXCR4, DGK, DGKA, DGKB, DGKD, DGKE, DGKG, DGKI, DGKK, DGKQ, DGKZ, DHFR, DNAM1 (CD226), EP2/4 receptors, adenosine receptors including A2AR, FAS, FASLG, GADS, GITR, GM-CSF, gp49B, HHLA2, HLA-A, HLA-B, HLA-C, HLA-DPA1, HLA-DPB1, HIV-LTR (long terminal repeat), HLA-DQA1, HLA-DQB1, HLA-DRA, HLA-DRB1, HLA-I, HVEM, HVEM, IA4, ICAM-1, ICOS, ICOS, ICOS (CD278), IFN-alpha/beta/gamma, IL-1 beta, IL-12, IL-15, IL-18, IL-23, IL2R beta, IL2R gamma, IL2RA, IL-6, IL7R alpha, ILT-2, ILT-4, ITGA4, ITGA4, ITGA6, ITGAD, ITGAE, ITGAL, ITGAM, ITGAX, ITGB1, ITGB2, ITGB7, KIR family receptors, KLRG1, LAIR-1, LAT, LIGHT, LTBR, Ly9 (CD229), MNK1/2, NKG2C, NKG2D, NKp30, NKp44, NKp46, NKp80 (KLRF1), OX2R, OX40, PAG/Cbp, PD-1, PD-L1, PD-L2, PGE2 receptors, PIR-B, PPP1R12C, PSGL1, PTPN2, RANCE/RANKL, ROSA26, SELPLG (CD162), SIRPalpha (CD47), SLAM (SLAMF1, SLAMF4 (CD244, 2B4), SLAMF5, SLAMF6 (NTB-A, Ly108), SLAMF7, SLP-76, TGFBR2, TIGIT, TIM-1, TIM-3, TIM-4, TMIGD2, TRA, TRAC, TRB, TRD, TRG, TNF, TNF-alpha, TNFR2, TUBA1, VISTA, VLA1, or VLA-6.

In some embodiments, the paired CRISPR nickase RNPs can be engineered to target an immune-related genomic locus listed in Table A.

TABLE A
Target genomic loci

| | | UniProtKB |
|---|---|---|
| | Gene | Identifier |
| Protein | Symbol | (human) |
| Programmed cell death-1 (PD-1) | PD-1 | Q15116 |
| Cluster of differentiation 52 (CD52) | CD52 | Q9UJ81 |
| Cytotoxic T-lymphocyte protein 4 (CTLA4) | CTLA4 | P16410 |
| Lymphocyte-activation protein 3 (LAG3) | LAG3 | P18627 |
| Integrin lymphocyte function-associated | ITGAL | P20701 |
| antigen 1 (LFA1) comprising integrin | ITGB2 | P05107 |
| alpha L chain (ITGAL) and integrin | | |
| beta 2 chain (ITGB2) | | |
| Hepatitis A virus cellular receptor 2 | HAVCR2 | Q8TDQ0 |
| (HAVCR2) (also called T-cell immuno- | | |
| globulin and mucin-domain containing-3, | | |
| TIM-3) | | |
| T-cell receptor alpha constant (TRAC) | TRAC | P01848 |
| T-cell receptor alpha locus (TCR-alpha) | TRA | A0A0C4ZLG8 |
| T-cell receptor beta locus (TCR-beta) | TRB | A0A0C4ZPA0 |

In one specific embodiment, the paired CRISPR nickase RNPs are engineered to target a PD-1 genomic locus. In another specific embodiment, the paired CRISPR nickase RNPs are engineered to target a CTLA4 genomic locus. In another specific embodiment, the paired CRISPR nickase RNPs are engineered to target a TIM-3 genomic locus. In another specific embodiment, the paired CRISPR nickase RNPs are engineered to target a TRAC genomic locus.

### (b) CRISPR Nickases

CRISPR nickases are derived from CRISPR nucleases by inactivation of one of the nuclease domains. In specific embodiments, the CRISPR nickase can be derived from a type II CRISPR nuclease. For example, the type II CRISPR nuclease can be a Cas9 protein. Suitable Cas9 nucleases include Streptococcus pyogenes Cas9 (SpCas9), Francisella novicida Cas9 (FnCas9), Staphylococcus aureus (SaCas9), Streptococcus thermophilus Cas9 (StCas9), Streptococcus pasteurianus (SpaCas9), Campylobacter jejuni Cas9 (CjCas9), Neisseria meningitis Cas9 (NmCas9), or Neisseria cinerea Cas9 (NcCas9). In other embodiments, the nickase can be derived from a type V CRISPR nuclease, such as a Cpf1 nuclease. Suitable Cpf1 nucleases include Francisella novicida Cpf1 (FnCpf1), Acidaminococcus sp. Cpf1 (AsCpf1), or Lachnospiraceae bacterium ND2006 Cpf1 (LbCpf1). In yet another embodiment, the nickase can be derived from a type VI CRISPR nuclease, e.g., Leptotrichia wadei Cas13a (LwaCas13a) or Leptotrichia shahii Cas13a (LshCas13a).

CRISPR nucleases comprise two nuclease domains. For example, a Cas9 nuclease comprises a HNH domain, which cleaves the guide RNA complementary strand, and a RuvC domain, which cleaves the non-complementary strand; a Cpf1 nuclease comprises a RuvC domain and a NUC domain; and a Cas13a nuclease comprises two HNEPN domains. When both nuclease domains are functional, CRISPR nuclease introduces a double-stranded break. Either nuclease domain can be inactivated by one or more mutations and/or deletions, thereby creating a variant that introduces a single-strand break in one strand of the double-stranded sequence. For example, one or more mutations in the RuvC domain of Cas9 nuclease (e.g., D10A, D8A, E762A, and/or D986A) results in an HNH nickase that nicks the guide RNA complementary strand; and one or more mutations in the HNH domain of Cas9 nuclease (e.g., H840A, H559A, N854A, N856A, and/or N863A) results in a RuvC nickase that nicks the guide RNA non-complementary strand. Comparable mutations can convert Cpf1 and Cas13a nucleases to nickases.

In specific embodiments, the CRISPR nickase can be a type II CRISPR nickase, a type V CRISPR nickase, or a type VI CRISPR nickase. For example, where the CRISPR nickase is a type II nickase, the CRISPR nickase can be a Cas9 nickase such as SpCas9, FnCas9, SaCas9, StCas9, SpaCas9, CjCas9, NmCas9, or NcCas9. By way of another example, where the CRISPR nickase is a type V nickase, the CRISPR nickase can be a Cpf1 nickase such as FnCpf1, AsCpf1, or LbCpf1. By way of yet another example, the CRISPR nickase can be a Cas13a nickase such as LwaCas13a or LshCas13a. It will be understood that the aforementioned CRISPR nickases will include the functionally relevant mutations in order to covert the nucleases to nickases, as described in the preceding paragraph. For example, the Cas9 nickase can be a Cas9-D10A nickase or a Cas9-H840A nickase. In one particular embodiment, the Cas9 nickase is a SpCas9-D10A nickase. In another particular embodiment, the Cas9 nickase is a SpCas9-H840A nickase.

The CRISPR nickase can further comprise at least one nuclear localization signal, at least one cell-penetrating domain, at least one marker domain, and/or at least one chromatin disrupting domain. The at least one nuclear localization signal, the at least one cell-penetrating domain, the at least one marker domain, and/or the at least one chromatin disrupting domain can be located at the N terminal end, C terminal end, and/or an internal location (provided the function of the CRISPR nickase is not affected).

Non-limiting examples of nuclear localization signals include PKKKRKV (SEQ ID NO:1), PKKKRRV (SEQ ID NO:2), KRPAATKKAGQAKKKK (SEQ ID NO:3), YGRKKRRQRRR (SEQ ID NO:4), RKKRRQRRR (SEQ ID NO:5), PAAKRVKLD (SEQ ID NO:6), RQRRNELKRSP (SEQ ID NO:7), VSRKRPRP (SEQ ID NO:8), PPKKARED (SEQ ID NO:9), PQPKKKPL (SEQ ID NO:10), SALIKKKKKMAP (SEQ ID NO:11), PKQKKRK (SEQ ID NO:12), RKLKKKIKKL (SEQ ID NO:13), REKKKFLKRR (SEQ ID NO:14), KRKGDEVDGVDEVAKKKSKK (SEQ ID NO:15), RKCLQAGMNLEARKTKK (SEQ ID NO:16), NQSSNFGPMKGGNFGGRSSGPYGGGGQYFAKPRNQGGY (SEQ ID NO:17), and RMRIZFKNKGKDTAELRRRRVEVSVELRKAKKDEQILKRRNV (SEQ ID NO:18).

Examples of suitable cell-penetrating domains include, without limit, GRKKRRQRRRPPQPKKKRKV (SEQ ID NO:19), PLSSIFSRIGDPPKKKRKV (SEQ ID NO:20), GALFLGWLGAAGSTMGAPKKKRKV (SEQ ID NO:21), GALFLGFLGAAGSTMGAWSQPKKKRKV (SEQ ID NO:22), KETWWETWWTEWSQPKKKRKV (SEQ ID NO:23), YARAAARQARA (SEQ ID NO:24), THRLPRRRRRR (SEQ ID NO:25), GGRRARRRRRR (SEQ ID NO:26), RRQRRTSKLMKR (SEQ ID NO:27), GWTLNSAGYLLGKINLKALAALAKKIL (SEQ ID NO:28), KALAWEAKLAKALAKALAKHLAKALAKALKCEA (SEQ ID NO:29), and RQIKIWFQNRRMKWKK (SEQ ID NO:30). Marker domains include fluorescent proteins and purification or epitope tags. Suitable fluorescent proteins include, without limit, green fluorescent proteins (e.g., GFP, eGFP, GFP-2, tagGFP, turboGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., BFP, EBFP, EBFP2, Azurite, mKalamal, GFPuv, Sapphire, T-sapphire), cyan fluorescent proteins (e.g., ECFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (e.g., mOrange, mKO, Kusabira-Orange, Monomeric Kusabira-Orange, mTangerine, tdTomato). Non-limiting examples of suitable purification or epitope tags include 6×His, FLAG®, HA, GST, Myc, and the like. Non-limiting examples of heterologous fusions which facilitate detection or enrichment of CRISPR complexes include streptavidin (Kipriyanov et al., Human Antibodies, 1995, 6(3):93-101), avidin (Airenne et al., Biomolecular Engineering, 1999, 16(1-4):87-92), monomeric forms of avidin (Laitinen et al., Journal of Biological Chemistry, 2003, 278(6):4010-4014), peptide tags which facilitate biotinylation during recombinant production (Cull et al., Methods in Enzymology, 2000, 326:430-440).

Examples of suitable chromatin disrupting domains include nucleosome interacting peptides derived from high mobility group (HMG) proteins (e.g., HMGB, HMGN proteins), the central globular domain of histone H1 variants (e.g., histone H1.0, H1.1, H1.2, H1.3, H1.4, H1.5, H1.6, H1.7, H1.8, H1.9, and H.1.10), or DNA binding domains of chromatin remodeling complexes (e.g., SWI/SNF, ISWI, CHD, Mi-2/NuRD, INO80, SWR1, or RSC complexes). In some instances, the chromatin disrupting domain can be HMGB1 box A domain, HMGB2 box A domain, HMGB3 box A domain, HMGN1 peptide, HMGN2 peptide, HMGN3 peptide, HMGN3 peptide, HMGN4 peptide, HMGN5 peptide, or human histone H1 central globular domain peptide.

The at least one nuclear localization signal, at least one cell-penetrating domain, at least one marker domain, and/or at least one chromatin disrupting domain can be linked directly to the CRISPR nickase via one or more chemical bonds (e.g., covalent bonds). Alternatively, the at least one nuclear localization signal, at least one cell-penetrating domain, at least one marker domain, and/or at least one chromatin disrupting domain or the one or more heterologous domains can be linked indirectly to the CRISPR nickase via one or more linkers. Suitable linkers include amino acids, peptides, nucleotides, nucleic acids, organic linker molecules (e.g., maleimide derivatives, N-ethoxybenzylimidazole, biphenyl-3,4′,5-tricarboxylic acid, p-aminobenzyloxycarbonyl, and the like), disulfide linkers, and polymer linkers (e.g., PEG). The linker can include one or more spacing groups including, but not limited to alkylene, alkenylene, alkynylene, alkyl, alkenyl, alkynyl, alkoxy, aryl, heteroaryl, aralkyl, aralkenyl, aralkynyl and the like. The linker can be neutral, or carry a positive or negative charge. Additionally, the linker can be cleavable such that the linker's covalent bond that connects the linker to another chemical group can be broken or cleaved under certain conditions, including pH, temperature, salt concentration, light, a catalyst, or an enzyme. In some embodiments, the linker can be a peptide linker. The peptide linker can be a flexible amino acid linker or a rigid amino acid linker. Additional examples of suitable linkers are well known in the art and programs to design linkers are readily available (Crasto et al., Protein Eng., 2000, 13(5):309-312).

In still other embodiments, the CRISPR nickase can be engineered by one or more amino acid substitutions, deletions, and/or insertions to have improved targeting specificity, improved fidelity, altered PAM specificity, decreased off-target effects, and/or increased stability. Non-limiting examples of one or more mutations that improve targeting specificity, improve fidelity, and/or decrease off-target effects include N497A, R661A, Q695A, K810A, K848A, K855A, Q926A, K1003A, R1060A, and/or D1135E (with reference to the numbering system of SpCas9).

### (c) Paired Guide RNAs

The paired CRISPR nickase RNPs comprise at least one pair of offset guide RNAs designed to hybridize with target sequences on opposite strands of a genomic locus of interest. A guide RNA comprises (i) a CRISPR RNA (crRNA) and (ii) a transacting crRNA (tracrRNA). The crRNA comprises a guide sequence at the 5′ end that is designed to hybridize with a target sequence (i.e., protospacer) in the genomic locus of interest. The target sequence is unique compared to the rest of the genome and is adjacent to a protospacer adjacent motif (PAM). The tracrRNA comprises sequences that interact with the CRISPR protein and the PAM sequence. While the guide sequence of each crRNA differs (i.e., is sequence specific), the tracrRNA sequence is generally the same in guide RNAs designed to complex with CRISPR proteins from a particular bacterial species.

The paired guide RNAs are engineered to hybridize with target sequences that are in close enough proximity to yield a double-stranded break upon two individual nicking events. The target region comprises the two target sequences and the adjacent PAM sequences. The pair of guide RNAs is configured such that the PAM sequences face outwards or are located at the distal ends of the target region (Ran et al., Cell, 2013, 154:1380-1389). Such a configuration is termed a “PAM-out” orientation. The distance between the two PAM sequences can range from about 30 base pairs (bp) to about 150 bp, from about 35 bp to about 120 bp, or from about 40 bp to about 80 bp. In various embodiments, the distance between the two PAM sequences can be about 35-40 bp, about 40-45 bp, about 45-50 bp, about 50-55 bp, about 55-60 bp, about 60-65 bp, about 65-70 bp, about 70-75 bp, about 75-80 bp, about 80-85 bp, about 85-90 bp, about 90-95 bp, or about 95-100 bp.

Each crRNA comprises a 5′ guide sequence that is complementary to a target sequence. In general, the complementarity between the crRNA guide sequence and the target sequence is at least 80%, at least 85%, at least 90%, at least 95%, or at least 99%. In specific embodiments, the complementarity is complete (i.e., 100%). In various embodiments, the length of the crRNA guide sequence can range from about 17 nucleotides to about 27 nucleotides. For example, the crRNA guide sequence can be about 17, 18, 19, 20, 21, 22, 23, 24, 25, 26, or 27 nucleotides in length. In some embodiments, the crRNA guide sequence can be about 19, 20, or 21 nucleotides in length. For example, the crRNA guide sequence can be 20 nucleotides long. In other embodiments, the crRNA guide sequence can be about 22, 23, or 24 nucleotides in length. For example, the crRNA guide sequence can be 23 nucleotides long. In one embodiment, the crRNA guide sequence comprises SEQ ID NO:31, SEQ ID NO:32, SEQ ID NO:33, or SEQ ID NO:34.

The target sequence is adjacent to a PAM sequence. CRISPR proteins from different bacterial species recognize different PAM sequences. For example, PAM sequences include 5′-NGG (SpCas9, FnCAs9), 5′-NGRRT (SaCas9), 5′-NNAGAAW (StCas9), 5′-NNNNGATT (NmCas9), 5-NNNNRYAC (CjCas9), and 5′-TTTV (Cpf1), wherein N is defined as any nucleotide, R is defined as either G or A, W is defined as either A or T, Y is defined an either C or T, and V is defined as A, C, or G. Cas9 PAMs are located 3′ of the target site, and cpf1 PAMs are located 5′ of the target site.

Each crRNA further comprises sequence at the 3′ end that is complementary to the 5′ end of the tracrRNA such that the 3′ end of the crRNA can hybridize with the 5′ end of the tracrRNA. The length of the 3′ sequence of the crRNA can range from about 6 to about 50 nucleotides, from about 15 to about 25 nucleotides. In various embodiments, the 3′ sequence of the crRNA ranges can be about 15, 16, 17, 18, 19, 20, 21, 22, 23, 24, or 25 nucleotides in length.

In addition to the sequence at the 5′ end of the tracrRNA that is complementary to the 3′ sequence of the crRNA, each tracrRNA further comprises 3′ repeat sequences that can form secondary structures (e.g., at least one stem loop, hairpin loop, etc.), which interact with the CRISPR protein. The sequence at the 3′ end of the tracrRNA remains single-stranded. In general, the tracrRNA sequence is based upon the wild type tracrRNA that interacts with a wild type CRISPR protein. Each tracrRNA can range in length from about 50 nucleotides to about 300 nucleotides. In various embodiments, the tracrRNA can range in length from about 50 to about 90 nucleotides, from about 90 to about 110 nucleotides, from about 110 to about 130 nucleotides, from about 130 to about 150 nucleotides, from about 150 to about 170 nucleotides, from about 170 to about 200 nucleotides, from about 200 to about 250 nucleotides, or from about 250 to about 300 nucleotides.

Each guide RNA can comprise two separate molecules, a crRNA and a tracrRNA. Alternatively, each guide RNA can be a single molecule in which the crRNA is linked to the tracrRNA. For example, a loop or a stem loop can be used to link the crRNA and the tracrRNA.

The guide RNAs can be synthesized chemically, enzymatically, or a combination thereof. For example, the guide RNAs can be synthesized using standard phosphoramidite-based solid-phase synthesis methods. Alternatively, the guide RNAs can be synthesized in vitro by operably linking DNA encoding the guide RNA to a promoter control sequence that is recognized by a phage RNA polymerase. Examples of suitable phage promoter sequences include T7, T3, SP6 promoter sequences, or variations thereof. In some embodiments, the crRNA is chemically synthesized and the tracrRNA is enzymatically synthesized.

Each guide RNA can comprise standard ribonucleotides and/or modified ribonucleotides. In some embodiments, the guide RNAs can comprise standard or modified deoxyribonucleotides. In embodiments in which the guide RNA is enzymatically synthesized, the guide RNA generally comprises standard ribonucleotides. In embodiments in which the guide RNA is chemically synthesized, the guide RNA can comprise standard or modified ribonucleotides and/or deoxyribonucleotides. Modified ribonucleotides and/or deoxyribonucleotides include base modifications (e.g., pseudouridine, 2-thiouridine, N6-methyladenosine, and the like) and/or sugar modifications (e.g., 2′-O-methy, 2′-fluoro, 2′-amino, locked nucleic acid (LNA), and so forth). The backbone of the guide RNA can also be modified to comprise phosphorothioate linkages, boranophosphate linkages, or peptide nucleic acids.

In other embodiments, the guide RNA can further comprise at least one detectable label. The detectable label can be a fluorophore (e.g., FAM, TMR, Cy3, Cy5, Texas Red, Oregon Green, Alexa Fluors, Halo tags, or suitable fluorescent dye), a detection tag (e.g., biotin, digoxigenin, and the like), quantum dots, or gold particles.

### (d) Specific Embodiments

In certain embodiments, the paired CRISPR nickase RNPs comprise (i) Cas9-D10A (+NLS) complexed with a guide RNA comprising SEQ ID NO:31 and (ii) Cas9-D10A (+NLS) complexed with a guide RNA comprising SEQ ID NO:32. In other embodiments, the paired CRISPR nickase RNPs comprise (i) Cas9-D10A (+NLS) complexed with a guide RNA comprising SEQ ID NO:33 and (ii) Cas9-D10A (+NLS) complexed with a guide RNA comprising SEQ ID NO:34. In other embodiments, the paired CRISPR nickase RNPs comprise (i) Cas9-D10A (+NLS) complexed with a guide RNA comprising SEQ ID NO:33 and (ii) Cas9-D10A (+NLS) complexed with a guide RNA comprising SEQ ID NO:32. In other embodiments, the paired CRISPR nickase RNPs comprise (i) Cas9-D10A (+NLS) complexed with a guide RNA comprising SEQ ID NO:39 and (ii) Cas9-D10A (+NLS) complexed with a guide RNA comprising SEQ ID NO:40. In other embodiments, the paired CRISPR nickase RNPs comprise (i) Cas9-D10A (+NLS) complexed with a guide RNA comprising SEQ ID NO:41 and (ii) Cas9-D10A (+NLS) complexed with a guide RNA comprising SEQ ID NO:42. In other embodiments, the paired CRISPR nickase RNPs comprise (i) Cas9-D10A (+NLS) complexed with a guide RNA comprising SEQ ID NO:43 and (ii) Cas9-D10A (+NLS) complexed with a guide RNA comprising SEQ ID NO:44. In other embodiments, the paired CRISPR nickase RNPs comprise (i) Cas9-D10A (+NLS) complexed with a guide RNA comprising SEQ ID NO:45 and (ii) Cas9-D10A (+NLS) complexed with a guide RNA comprising SEQ ID NO:46. In other embodiments, the paired CRISPR nickase RNPs comprise (i) Cas9-D10A (+NLS) complexed with a guide RNA comprising SEQ ID NO:47 and (ii) Cas9-D10A (+NLS) complexed with a guide RNA comprising SEQ ID NO:48. In other embodiments, the paired CRISPR nickase RNPs comprise (i) Cas9-D10A (+NLS) complexed with a guide RNA comprising SEQ ID NO:49 and (ii) Cas9-D10A (+NLS) complexed with a guide RNA comprising SEQ ID NO:50. In other embodiments, the paired CRISPR nickase RNPs comprise (i) Cas9-D10A (+NLS) complexed with a guide RNA comprising SEQ ID NO:51 and (ii) Cas9-D10A (+NLS) complexed with a guide RNA comprising SEQ ID NO:52. In other embodiments, the paired CRISPR nickase RNPs comprise (i) Cas9-D10A (+NLS) complexed with a guide RNA comprising SEQ ID NO:53 and (ii) Cas9-D10A (+NLS) complexed with a guide RNA comprising SEQ ID NO:54. In other embodiments, the paired CRISPR nickase RNPs comprise (i) Cas9-D10A (+NLS) complexed with a guide RNA comprising SEQ ID NO:55 and (ii) Cas9-D10A (+NLS) complexed with a guide RNA comprising SEQ ID NO:56.

### (II) Kits

A further aspect of the present disclosure provides kits comprising paired CRISPR nickase RNPs as described above in section (I). In some embodiments, the CRISPR nickase can be complexed with each of the paired guide RNAs and provided as RNPs ready for use. In other embodiments, the CRISPR nickase and each of the paired guide RNAs can be provided separately for the end user to complex into RNPs prior to use. The kits can further comprise transfection reagents, cell growth media, selection media, reaction buffers, and the like. In some embodiments, the kits can further comprise one or more donor polynucleotides for gene conversion/correction of a genomic locus of interest. The kits provided herein generally include instructions for carrying out the methods detailed below. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### (III) Methods for Efficient Modification of Immune-Related Genomic Loci

Another aspect of the present disclosure encompasses methods for efficiently modifying a genomic locus in a eukaryotic cell. The method comprises introducing paired CRISPR nickase RNPs as described above in section (I) into the cell, wherein the CRISPR nickases coordinately introduce a double-stranded break into the targeted genomic locus such that cellular repair of the double-stranded break leads to modification of the genomic locus.

The double-stranded break can be repaired by nonhomologous end joining (NHEJ) such that there is an insertion of at least one nucleotide and/or a deletion of at least one nucleotide (i.e., indels) and the genomic locus is inactivated. For example, the genomic locus can be knocked-down (i.e., monoallelic mutation) and produce a reduced amount of gene product, or knocked-out (i.e., biallelic mutation) and produce no gene product.

In some embodiments, the method further comprises introducing into the eukaryotic cell a donor polynucleotide comprising a donor sequence having at least one nucleotide change relative to the target region of the genomic locus of interest, wherein repair of the double-stranded break by homology-directed repair (HDR) results in integration or exchange of the donor sequence such that the genomic locus of interest is modified by at least one nucleotide substitution (e.g., gene correction/conversion).

The methods disclosed herein comprise introducing CRISPR nickase RNPs into the cell, as opposed to nucleic acids encoding the CRISPR components. Thus, the CRISPR nickase RNPs can immediately cleave the target genomic locus, and the cell does not have to transcribe/translate the CRISPR components. Since foreign proteins and RNAs tends to be rapidly degraded, the CRISPR nickase RNPS have transient effects. Moreover, the delivery of CRISPR nickase RNPs avoids the prolonged expression problems observed when nucleic acids encoding the CRISPR components are introduced into cells (Kim et al., Genome Research, 2014, 24(6):1012-1019).

In general, the utilization of paired CRISPR nickase RNPs results in high frequency of genome modifications. As detailed in Example 4, in human primary T-cells, the paired Cas9 nickase RNPs generated indel frequency of 29% at the CTLA-4 locus, 11% at the TIM-3 locus, and 14% at the TRAC locus, as estimated using TIDE/ICE (Tracking of Indels by Decomposition/Inference of CRISPR Edits) assay. Often, the utilization of paired CRISPR nickase RNPs results in an increased frequency of genome modifications as compared to the utilization of a single CRISPR nuclease RNP. As detailed in Example 1, in K562 cells, the paired Cas9 nickase RNPs generated an average indel frequency of 21% at the PD-1 locus, whereas the Cas9 nuclease RNP resulted in an average indel frequency of 9.5% at the PD-1 locus, as estimated with a CEL-1 nuclease assay. Similarly, as detailed in Example 2, in human primary T cells, the paired Cas9 nickase RNPs generated an average indel frequency of 5.6% at the PD-1 locus, whereas the Cas9 nuclease RNP resulted in an average indel frequency of 1.6% at the PD-1 locus, as estimated using next generation sequencing. As detailed in Example 4, the paired Cas9 nickase RNPs generated indel frequency of 11% at the TIM-3 locus, whereas the Cas9 nuclease RNP resulted in indel frequency of 4% at the TIM-3 locus, as estimated using TIDE/ICE assay.

### (a) Introduction into the Cell

The method comprises introducing paired CRISPR nickase RNAs into the cell. In some embodiments, the CRISPR nickase and each of the paired guide RNAs can be complexed into an RNP immediately prior to delivery to the cell. In other embodiments, the CRISPR nickase and each of the paired guide RNAs can be complexed (and stored appropriately) for hours, days, weeks, or months prior to delivery to the cell.

In general, the molar ratio of the pair of guide RNAs to CRISPR nickase can range from about 0.1:1 to about 100:1. Thus, for example, the molar ratio of the pair guide RNAs to CRISPR nickase can be 0.25:1, 0.5:1, 0.75:1, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1. 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, or 100:1. In some embodiments, the molar ratio of the pair of guide RNAs to CRISPR nickase is from about 0.5:1 to about 50:1. In some embodiments, the molar ratio of the pair of guide RNAs to CRISPR nickase is from about 1:1 to about 75:1. In some embodiments, the molar ratio of the pair of guide RNAs to CRISPR nickase is from about 1:1 to about 25:1. In some embodiments, the molar ratio of the pair of guide RNAs to CRISPR nickase is from about 1:1 to about 15:1. In some embodiments, the molar ratio of the pair of guide RNAs to CRISPR nickase is from about 1:1 to about 10:1. In some embodiments, the molar ratio of the pair of guide RNAs to CRISPR nickase is from about 2:1 to about 10:1. In other embodiments, the molar ratio of the pair of guide RNAs to CRISPR nickase is 0.5:1, 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1.

The CRISPR nickase RNPs can be delivered to the cell by a variety of means. In some embodiments, the CRISPR nickase RNPs can be introduced into the cell via a suitable transfection method. For example, the CRISPR nickase RNPs can be introduced with an electroporation-based transfection procedure, i.e., nucleofection. Nucleofection methods and apparatuses are well known in the art. In other embodiments, the CRISPR nickase RNPs can be introduced in the cell by incubation in the presence of an endosomolytic agent such as a cell penetrating peptide or derivative thereof (Erazo-Oliverase et al., Nature Methods, 2014, 11:861-867). In yet other embodiments, the CRISPR nickase RNPs can be introduced in the cell by microinjection.

In general, the cell is maintained under conditions appropriate for cell growth and/or maintenance. Suitable cell culture conditions are well known in the art and are described, for example, in Santiago et al., Proc. Natl. Acad. Sci. USA, 2008, 105:5809-5814; Moehle et al., Proc. Natl. Acad. Sci. USA, 2007, 104:3055-3060; Urnov et al., Nature, 2005, 435:646-651; and Lombardo et al., Nat. Biotechnol., 2007, 25:1298-1306. Those of skill in the art appreciate that methods for culturing cells are known in the art and can and will vary depending on the cell type. Routine optimization may be used, in all cases, to determine the best techniques for a particular cell type.

### (b) Optional Donor Polynucleotide

In some embodiments, the method further comprises intruding into the cell at least one donor polynucleotide comprising a donor sequence having at least one nucleotide change relative to the target region of the genomic locus of interest. Thus, upon integration or exchange with the native genomic sequence, the modified genomic locus comprises at least one nucleotide change such that the cell produces a modified gene product.

The donor sequence comprises at least one nucleotide change relative to the target region of the genomic locus. As such, the donor sequence has substantial sequence identity to the target region in the genomic locus of interest. Depending upon the length of the target region, the donor sequence can be flanked by sequences having substantial sequence identity to sequences located upstream and downstream of the target region. As used herein, the phrase “substantial sequence identity” refers to sequences having at least about 75% sequence identity. Thus, the donor sequence (and optional flanking sequences) in the donor polynucleotide can have about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the genomic locus of interest. In specific embodiments, the optional flanking sequences can have about 95% or 100% sequence identity with corresponding sequences in the genomic locus of interest.

The length of the donor sequence (and optional flanking sequences) can and will vary. For example, the donor sequence (and optional flanking sequences) can range in length from about 30 nucleotides to about 1000 nucleotides. In certain embodiments, the donor sequence (and optional flanking sequences) can range from about 30 nucleotides to about 100 nucleotides, from about 100 nucleotides to about 300 nucleotides, or from about 300 nucleotides to about 10000 nucleotides in length.

The donor polynucleotide can be single-stranded or double-stranded, linear or circular, and/or RNA or DNA. In some embodiments, the donor polynucleotide can be a vector, e.g., a plasmid vector. In other embodiments, the donor polynucleotide can be a single-stranded oligonucleotide.

### (c) Cell Types

The method comprises introducing the paired CRISPR nickase RNPs into a eukaryotic cell. The eukaryotic cell can be a human cell or an animal cell. In most embodiments, the eukaryotic cell will be an immune cell. Suitable immune cells include lymphocytes, such as T-cells (e.g., killer T-cells, helper T-cells, gamma delta T-cells), B-cells (e.g., pro B-cells, memory B cells, plasma cells), or natural killer (NK) cells, neutrophils, monocytes/macrophages, granulocytes, mast cells, and dendritic cells. In some embodiments, the cell can be a non-immune cell. The eukaryotic cell can be a primary cell or a cell line cell. In particular embodiments, the cell can be a human primary T-cell.

### (IV) Applications

The compositions and methods disclosed herein can be used in a variety of therapeutic, diagnostic, industrial, and research applications. In some embodiments, the present disclosure can be used to develop, test, and/or implement immuno-oncology, cancer immunotherapy, immunotherapy, immune therapeutics, immunodiagnostics, or other immune based treatments. For example, specific compositions can be engineered to target specific types of breast cancers (e.g., ER-positive, PR-positive, triple negative, etc.), prostate cancers, lung cancers, skin cancers, etc.

In other embodiments, the present disclosure can be used to modify genomic loci of interest in a cell or animal in order to model and/or study the function of genes, study genetic or epigenetic conditions of interest, or study biochemical pathways involved in various diseases or disorders. For example, transgenic animals can be created that model diseases or disorders, wherein the expression of one or more nucleic acid sequences associated with a disease or disorder is altered. The disease model can be used to study the effects of mutations on the animal, study the development and/or progression of the disease, study the effect of a pharmaceutically active compound on the disease, and/or assess the efficacy of a potential gene therapy strategy.

In other embodiments, the compositions and methods can be used to perform efficient and cost effective functional genomic screens, which can be used to study the function of genes involved in a particular biological process and how any alteration in gene expression can affect the biological process, or to perform saturating or deep scanning mutagenesis of genomic loci in conjunction with a cellular phenotype. Saturating or deep scanning mutagenesis can be used to determine critical minimal features and discrete vulnerabilities of functional elements required for gene expression, drug resistance, and reversal of disease, for example.

### (IV) Methods of Treatment

In another aspect, a method of treating a subject, e.g., reducing or ameliorating, a hyperproliferative condition or disorder (e.g., a cancer), e.g., solid tumor, a soft tissue tumor, or a metastatic lesion, in a subject is provided. The method includes modifying a cell in accordance with the methods described herein, typically ex vivo, and delivering or administering to a subject in need of treatment the modified cells, alone or in combination with other agents or therapeutic modalities.

For example, the modification regime targeted to a locus (protein coding gene, non-coding gene, safe harbor locus, or other) within the human genome to knockdown, knockout, or knockin a particular target gene(s). By inactivating a gene it is intended that the gene of interest is not expressed in a functional protein or RNA form (i.e., knockout). Alternatively, the gene of interest may be modified such that its expression and/or functionality is reduced (i.e., knockdown). By way of another alternative, an exogenous or donor sequence may be copied or integrated into the genomic sequence (i.e., knockin or integration). For example, a corrected version of a mutated or otherwise faulty gene may be introduced by correction of a small endogenous gene region (such as a single nucleic acid change, or several nucleic acid changes) or the functional replacement of an entire gene by introduction of a synthetic copy which results in disease treatment. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art.

Cancer treatment as described herein is meant to include all types of cancerous growths or oncogenic processes, metastatic tissues or malignantly transformed cells, tissues, or organs, irrespective of histopathologic type or stage of invasiveness. Examples of cancerous disorders include, but are not limited to, solid tumors, hematological cancers, soft tissue tumors, and metastatic lesions.

Examples of solid tumors include malignancies, e.g., sarcomas, and carcinomas (including adenocarcinomas; and squamous cell carcinomas), of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. Squamous cell carcinomas include malignancies, e.g., in the lung, esophagus, skin, head and neck region, oral cavity, anus, and cervix. Metastatic lesions of the aforementioned cancers can also be treated or prevented using the methods and compositions of the disclosure.

Exemplary cancers whose growth can be inhibited using the methods nad compositions disclosed herein include cancers typically responsive to immunotherapy. Non-limiting examples of preferred cancers for treatment include lymphoma (e.g., diffuse large B-cell lymphoma, Hodgkin lymphoma, non-Hodgkin's lymphoma), breast cancer (e.g., metastic breast cancer), lung cancer (e.g., non-small cell lung cancer (NSCLC), e.g., stage IV or recurrent non-small cell lung cancer, a NSCLC adenocarcinoma, or a NSCLC squamous cell carcinoma), myeloma (e.g., multiple myeloma), leukemia (e.g., chronic myelogenous leukemia), skin cancer (e.g., melanoma (e.g., stage III or IV melanoma) or Merkel cell carcinoma), head and neck cancer (e.g., head and neck squamous cell carcinoma (HNSCC)), myelodysplastic syndrome, bladder cancer (e.g., transitional cell carcinoma), kidney cancer (e.g., renal cell cancer, e.g., clear-cell renal cell carcinoma, e.g., advanced or metastatic clear-cell renal cell carcinoma), and colon cancer. Additionally, refractory or recurrent malignancies can be treated using the antibody molecules described herein.

Examples of other cancers that can be treated include bone cancer, pancreatic cancer, skin cancer, cancer of the head or neck, cutaneous or intraocular malignant melanoma, uterine cancer, ovarian cancer, rectal cancer, anal cancer, gastro-esophageal, stomach cancer, testicular cancer, uterine cancer, carcinoma of the fallopian tubes, carcinoma of the endometrium, carcinoma of the cervix, carcinoma of the vagina, carcinoma of the vulva, Merkel cell cancer, Hodgkin lymphoma, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, multiple myeloma, myelodisplastic syndromes, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos (e.g., mesothelioma), and combinations of said cancers.

In one embodiment, the tumor or cancer is chosen from adenoma, angio-sarcoma, astrocytoma, epithelial carcinoma, germinoma, glioblastoma, glioma, hamartoma, hemangioendothelioma, hemangiosarcoma, hematoma, hepato-blastoma, leukemia, lymphoma, medulloblastoma, melanoma, neuroblastoma, osteosarcoma, retinoblastoma, rhabdomyosarcoma, sarcoma, and teratoma. The tumor can be chosen from acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangio-carcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic, papillary serous adeno-carcinoma, pineal cell, pituitary tumors, plasmacytoma, pseudo-sarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

Thus, for example, the present disclosure provides methods for the treatment of a variety of cancers, including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyoscarcoma, and osteosarcoma; and other tumors including melanoma, xenoderma pigmentosum, keratoactanthoma, seminoma, thyroid follicular cancer, and teratocarcinoma.

For example, particular leukemias that can be treated with the compositions and methods described herein include, but are not limited to, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Lymphomas can also be treated with the compositions and methods described herein. Lymphomas are generally neoplastic transformations of cells that reside primarily in lymphoid tissue. Lymphomas are tumors of the immune system and generally are present as both T cell- and as B cell-associated disease. Among lymphomas, there are two major distinct groups: non-Hodgkin's lymphoma (NHL) and Hodgkin's disease. Bone marrow, lymph nodes, spleen and circulating cells, among others, may be involved. Treatment protocols include removal of bone marrow from the patient and purging it of tumor cells, often using antibodies directed against antigens present on the tumor cell type, followed by storage. The patient is then given a toxic dose of radiation or chemotherapy and the purged bone marrow is then re-infused in order to repopulate the patient's hematopoietic system.

Other hematological malignancies that can be treated with the compositions and methods described herein include myelodysplastic syndromes (MDS), myeloproliferative syndromes (MPS) and myelomas, such as solitary myeloma and multiple myeloma. Multiple myeloma (also called plasma cell myeloma) involves the skeletal system and is characterized by multiple tumorous masses of neoplastic plasma cells scattered throughout that system. It may also spread to lymph nodes and other sites such as the skin. Solitary myeloma involves solitary lesions that tend to occur in the same locations as multiple myeloma.

Cells that are targeted for use in the treatment methods described herein can include, for example, T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

The administration of the cells or population of cells modified according to the present disclosure may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, by intravenous or intralymphatic injection, or intraperitoneally. In one embodiment, the modified cells of the present disclosure are preferably administered by intravenous injection.

In one embodiment, any of the targets described herein are modulated in CAR T cells before administering to a patient in need thereof.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

In some embodiments, the method can further comprise administration of one or more additional agents (e.g., combination therapy). For example, one or more additional agents may be administered to the subject in conjunction with (e.g., before, after, or simultaneous with the treatment described herein) including chemotherapeutic agents, anti-angiogenesis agents and agents that reduce immune-suppression.

The therapeutic agent can be, for example, a chemotherapeutic or biotherapeutic agent, radiation, or immunotherapy. Any suitable therapeutic treatment for a particular cancer may be administered. Examples of chemotherapeutic and biotherapeutic agents include, but are not limited to, an angiogenesis inhibitor, such ashydroxy angiostatin K1-3, DL-α-Difluoromethyl-ornithine, endostatin, fumagillin, genistein, minocycline, staurosporine, and thalidomide; a DNA intercaltor/cross-linker, such as Bleomycin, Carboplatin, Carmustine, Chlorambucil, Cyclophosphamide, cis-Diammineplatinum(II) dichloride (Cisplatin), Melphalan, Mitoxantrone, and Oxaliplatin; a DNA synthesis inhibitor, such as (±)-Amethopterin (Methotrexate), 3-Amino-1,2,4-benzotriazine 1,4-dioxide, Aminopterin, Cytosine β-D-arabinofuranoside, 5-Fluoro-5′-deoxyuridine, 5-Fluorouracil, Ganciclovir, Hydroxyurea, and Mitomycin C; a DNA-RNA transcription regulator, such as Actinomycin D, Daunorubicin, Doxorubicin, Homoharringtonine, and Idarubicin; an enzyme inhibitor, such as S(+)-Camptothecin, Curcumin, (−)-Deguelin, 5,6-Dichlorobenzimidazole 1-β-D-ribofuranoside, Etoposide, Formestane, Fostriecin, Hispidin, 2-Imino-1-imidazoli-dineacetic acid (Cyclocreatine), Mevinolin, Trichostatin A, Tyrphostin AG 34, and Tyrphostin AG 879; a gene regulator, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid all trans (Vitamin A acid), 9-cis-Retinoic Acid, 13-cis-Retinoic acid, Retinol (Vitamin A), Tamoxifen, and Troglitazone; a microtubule inhibitor, such as Colchicine, docetaxel, Dolastatin 15, Nocodazole, Paclitaxel, Podophyllotoxin, Rhizoxin, Vinblastine, Vincristine, Vindesine, and Vinorelbine (Navelbine); and unclassified therapeutic agents, such as 17-(Allylamino)-17-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-α, Rapamycin, Sex hormone-binding globulin, Thapsigargin, and Urinary trypsin inhibitor fragment (Bikunin). The therapeutic agent may be altretamine, amifostine, asparaginase, capecitabine, cladribine, cisapride, cytarabine, dacarbazine (DTIC), dactinomycin, dronabinol, epoetin alpha, filgrastim, fludarabine, gemcitabine, granisetron, ifosfamide, irinotecan, lansoprazole, levamisole, leucovorin, megestrol, mesna, metoclopramide, mitotane, omeprazole, ondansetron, pilocarpine, prochloroperazine, or topotecan hydrochloride.

The therapeutic agent can also be a monoclonal antibody such as 131I-tositumomab, 90Y-ibritumomab tiuxetan, ado-trastuzumab emtansine (Kadcyla™), ado-trastuzumab emtansine, afatinib dimaleate (Gilotrif®), alemtuzumab (Campath®), axitinib (Inlyta®), Bevacizumab (Avastin®), bortezomib (Velcade®), bosutinib (Bosulif®), brentuximab vedotin (Adcetris®), Cabozantinib (Cometriq™), carfilzomib (Kyprolis®), ceritinib (LDK378/Zykadia), Cetuximab (Erbitux®), crizotinib (Xalkori®), dabrafenib (Tafinlar®), dasatinib (Sprycel®), Denosumab (Xgeva®), erlotinib (Tarceva®), erlotinib (Tarceva®), gefitinib (Iressa®), ibritumomab tiuxetan (Zevalin®), ibrutinib (Imbruvica™), idelalisib (Zydelig®), imatinib mesylate (Gleevec®), lapatinib (Tykerb®), nilotinib (Tasigna®), obinutuzumab (Gazyva™), ofatumumab (Arzerra®), panitumumab (Vectibix®), pazopanib (Votrient®), pembrolizumab (Keytruda®), pertuzumab (Perjeta™), Ramucirumab (Cyramza™), regorafenib (Stivarga®), rituximab (Rituxan®), siltuximab (Sylvant™), sorafenib (Nexavar®), sunitinib (Sutent®), Tositumomab and 131 I-tositumomab (Bexxar®), trametinib (Mekinist®), trastuzumab (Herceptin®), vandetanib (Caprelsa®), Vemurafenib (Zelboraf®), and Vismodegib (Erivedge™).The therapeutic agent can also be a neoantigen.

The therapeutic agent may be a cytokine such as interferons (INFs), interleukins (ILs), or hematopoietic growth factors. For example, the therapeutic agent can be INF-α, IL-2, Aldesleukin, IL-2, Erythropoietin, Granulocyte-macrophage colony-stimulating factor (GM-CSF) or granulocyte colony-stimulating factor.

The therapeutic agent may be a targeted therapy such as abiraterone acetate (Zytiga®), Alitretinoin (Panretin®), anastrozole (Arimidex®), belinostat (Beleodaq™), bexarotene (Targretin®), Cabazitaxel (Jevtana®), denileukin diftitox (Ontak®), enzalutamide (Xtandi®), everolimus (Afinitor®), exemestane (Aromasin®), fulvestrant (Faslodex®), lenaliomide (Revlimid®), lenaliomide (Revlimid®), letrozole (Femara®), pomalidomide (Pomalyst®), pralatrexate (Folotyn®), radium 223 chloride (Xofigo®), romidepsin (Istodax®), temsirolimus (Torisel®), toremifene (Fareston®), Tretinoin (Vesanoid®), vorinostat (Zolinza®), and ziv-aflibercept (Zaltrap®). Additionally, the therapeutic agent may be an epigenetic targeted drug such as HDAC inhibitors, kinase inhibitors, DNA methyltransferase inhibitors, histone demethylase inhibitors, or histone methylation inhibitors. The epigenetic drugs may be Azacitidine (Vidaza), Decitabine (Dacogen), Romidepsin (Istodax), Ruxolitinib (Jakafi), or Vorinostat (Zolinza).

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd Ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “about” when used in relation to a numerical value, x, for example means x±5%.

As used herein, the terms “complementary” or “complementarity” refer to the association of double-stranded nucleic acids by base pairing through specific hydrogen bonds. The base pairing may be standard Watson-Crick base pairing (e.g., 5′-A G T C-3′ pairs with the complementary sequence 3′-T C A G-5′). The base pairing also may be Hoogsteen or reversed Hoogsteen hydrogen bonding. Complementarity is typically measured with respect to a duplex region and thus, excludes overhangs, for example. Complementarity between two strands of the duplex region may be partial and expressed as a percentage (e.g., 70%), if only some (e.g., 70%) of the bases are complementary. The bases that are not complementary are “mismatched.” Complementarity may also be complete (i.e., 100%), if all the bases in the duplex region are complementary.

A “gene,” as used herein, refers to a chromosomal region (including exons and introns) encoding a gene product, as well as all chromosomal regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions. A “genomic locus” refers to a position on a chromosome comprising the gene sequence.

The term “nickase” refers to an enzyme that cleaves one strand of a double-stranded nucleic acid sequence (i.e., nicks a double-stranded sequence). For example, a nuclease with double strand cleavage activity can be modified by mutation and/or deletion to function as a nickase and cleave only one strand of a double-stranded sequence.

The term “nuclease,” as used herein, refers to an enzyme that cleaves both strands of a double-stranded nucleic acid sequence.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “nucleotide” refers to deoxyribonucleotides or ribonucleotides. The nucleotides may be standard nucleotides (i.e., adenosine, guanosine, cytidine, thymidine, and uridine), nucleotide isomers, or nucleotide analogs. A nucleotide analog refers to a nucleotide having a modified purine or pyrimidine base or a modified ribose moiety. A nucleotide analog may be a naturally occurring nucleotide (e.g., inosine, pseudouridine, etc.) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

The term “subject” and “individual” are used interchangeably herein, and refer to an animal, for example a human, to whom treatment, including prophylactic treatment, with a composition according to the present invention, is provided. The term “subject” as used herein refers to human and non-human animals. The term “non-human animals” includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is a non-human mammal. In another embodiment, the subject is human. In another embodiment, the subject is an experimental animal or animal substitute as a disease model. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species.

The terms “target sequence” and “target site” are used interchangeably to refer to the specific sequence in the genomic locus of interest to which a CRISPR RNP is targeted.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website.

As various changes could be made in the above-described cells and methods without departing from the scope of the invention, it is intended that all matter contained in the above description and in the examples given below, shall be interpreted as illustrative and not in a limiting sense.

### EXAMPLES

The following examples illustrate certain aspects of the disclosure.

### Example 1. Evaluation of CRISPR Nickase RNPs on PD-1 in K562 Cells

Programmed cell death-1 (PD-1 or PCD-1), a cell surface receptor, is a potential target for checkpoint blockade in cancer immunotherapy. Sets of paired crRNAs were designed for CRISPR-nickase RNPs on PD-1 (Table 1). The paired crRNAs were configured in the PAM-out orientation.

TABLE 1
Design of Paired crRNAs on PD-1

| | | | SEQ |
|---|---|---|---|
| Design | crRNA | Sequence | ID NO: |
| #1 | crRNA-a | GCGTGACTTCCACATGAGCGTGG | 31 |
| | crRNA-b | GCAGTTGTGTGACACGGAAGCGG | 32 |
| #2 | crRNA-c | GACAGCGGCACCTACCTCTGTGG | 33 |
| | crRNA-d | GGGCCCTGACCACGCTCATGTGG | 34 |
| #3 | crRNA-c | GACAGCGGCACCTACCTCTGTGG | 33 |
| | crRNA-b | GCAGTTGTGTGACACGGAAGCGG | 32 |

SpCas9-D10A nickase RNPs containing paired crRNA designs #1, #2, or #3 were tested and compared with SpCas9 nuclease RNPs containing individual crRNAs (crRNA-a, crRNA-b, crRNA-c, or crRNA-d). To form the RNPs, each of Cas9 protein (+NLS), tracrRNA and crRNA was resuspended to a concentration of 30 μM in either the supplied resuspension solution or 10 mM Tris buffer with a pH of 7.5. They were then assembled in an 11 μL mix at a molar ratio of 5:5:1 (crRNA:tracrRNA:Cas9 protein) and left at room temperature for 5 minutes immediately before use. For the nickase RNPs, two RNPs were formed separately and added to the cells simultaneously immediately before transfection. Transfection was done using a nucleofector system (Lonza) with the entire RNP mix added to 100 μL of K562 cells (approximately 350K cells).

Genomic DNA was extracted from the K562 cells using a DNA Extraction Solution (Epicentre), and the target sites were PCR amplified (Forward PD-1 primer: 5′-GGACAACGCCACCTTCACCTGC, SEQ ID NO:35 Reverse PD-1 primer: 5′-CTACGACCCTGGAGCTCCTGAT; SEQ ID NO:36. The CEL-1 Assay was performed using the Surveyor Mutation Detection Kit (IDT). First, the PCR amplicons went through a denaturing and annealing step in the thermocycler after amplification to form a heteroduplex, followed by a digestion with the Nuclease and Enhancer proteins at 42° C. before being electrophoresed on a 10% TBE Gel (Thermofisher). The gel was then stained in 100 ml 1×TBE buffer with 2 μL of 10 mg/ml ethidium bromide for 5 min, then washed with 1×TBE buffer and visualized with a UV illuminator. The resulting bands were analyzed using Image J software. Table 2 presents the results.

TABLE 2
Genome editing on PD-1 with Cas9 Nuclease
RNPs or Dual Cas9 Nickase RNPs

| Condition | Indel % |
|---|---|
| SpCas9 nuclease + tracrRNA + crRNA-a | 11 |
| SpCas9 nuclease + tracrRNA + crRNA-b | 13 |
| SpCas9 nuclease + tracrRNA + crRNA-c | 12 |
| SpCas9 nuclease + tracrRNA + crRNA-d | 2 |
| SpCas9 nickase + tracrRNA + crRNA-a + crRNA-b | 22 |
| SpCas9 nickase + tracrRNA + crRNA-c + crRNA-d | 24 |
| SpCas9 nickase + tracrRNA + crRNA-c + crRNA-b | 17 |
| Control | 0 |

As shown in Table 2, successful genome editing on PD-1 was generated with SpCas9 nickase RNPs in K562 cells. Surprisingly, SpCas9 nickase RNPs' genome editing efficiencies on PD-1 were much higher than those of SpCas9 nuclease RNPs. For example, SpCas9 nickase RNPs design #1, that contains crRNA-a and crRNA-b, resulted in 22% indels; while SpCas9 nuclease RNPs with crRNA-a or crRNA-b lead to only 11% or 13% indels, respectively.

### Example 2. Evaluation of CRISPR Nickase RNPs on PD-1 in Primary T Cells

SpCas9 nuclease RNPs and SpCas9 nickase RNPs were prepared as described in Example 1. CD8+ human primary T cells (AllCells, LLC) were maintained in a T cell expansion medium (Sigma-Aldrich) supplemented with 10% human AB serum (Sigma-Aldrich), 1×L-glutamine alternative (Gibco), 8 ng/mL IL-2 (Gibco), and 50 μM mercaptoethanol (Sigma). Cells were stimulated with T cell expansion beads (i.e., DYNABEADS™ Human T-Expander CD3/CD28; Gibco) 7 days prior to nucleofection. CD8+ human primary T cells (approximately 500 K cells) per transfection were used and the transfection was done using the nucleofection system as described in Example 1. Cells were cultured in the presence of the T cell expansion beads.

The editing efficiencies of SpCas9 nickase RNPs and SpCas9 nuclease RNPs were measured by using next generation sequencing (NGS). Six days post nucleofection, PCR was performed using a Taq reaction mixture (JUMPSTART™ REDTAQ® READYMIX™ Reaction Mix; Sigma-Aldrich) and primers that flanked the genomic cut site. The primers were tagged with partial IIlumina adapter sequences

NickFOR-ILLUMIPD1:
(SEQ ID NO: 37)
TCGTCGGCAGCGTCAGATGTGTATAAGAGACAGNNNNNNGGACAACGCCA
CCTTCACCTG
NickREV-ILLUMIPD1:
(SEQ ID NO: 38)
GTCTCGTGGGCTCGGAGATGTGTATAAGAGACAGNNNNNNCTACGACCCT
GGAGCTCCTGAT.

The thermal cycling conditions included a heat denaturing step at 95° C. for 5 minutes followed by 34 cycles of 95° C. for 30 seconds, anneal at 67.7° C. for 30 seconds, and extension at 70° C. for 30 seconds. Amplification was followed by a final extension at 70° C. for 10 minutes and a cool down to 4° C.

A limited-cycle PCR was carried out to index the amplified PCR product. A total reaction volume of 50 μL included 25 μL of the Taq reaction mix mentioned above, 5 μL of amplified PCR product, 10 μL H₂O, and 5 μL each of 5 μM Nextera XT Index 1 (i7) and Index 2 (i5) oligos. The thermal cycling conditions consisted of an initial heat denature at 95° C. for 3 minutes, followed by 8 cycles of 95° C. for 30 seconds, 55° C. for 30 seconds, and 72° C. for 30 seconds. A final extension was carried out at 72° C. for 5 minutes and the reaction was cooled down to 4° C. PCR purification was carried out using magnetic PCR purification beads (Corning), using 25 μL of indexed sample at a 8:1 bead to PCR ratio. DNA was eluted in 25 μL of 10 mM Tris.

PicoGreen fluorescent dye (Invitrogen) was used for quantification of indexed samples. Purified indexed PCR was diluted to 1:100 with 1×TE. PicoGreen was diluted to 1:200 with 1×TE. Equal volume of diluted PicoGreen was added to the diluted indexed PCR sample yielding a final 1:1 dilution ratio in a fluorescence plate reader. Samples were excited at 475 nm and read at 530 nm. All samples were normalized to 4 nM with 1×TE, and 64 of each normalized sample was collected and pooled.

Stock 10 M NaOH was serially diluted with H₂O to yield a final concentration of 0.1 M on the day of library preparation. To denature the DNA, 5 μL of 0.1 M NaOH and 5 μL of the pooled 4 nM library were mixed together and incubated at room temperature for 5 minutes. To this was added 9904 of cold Illumina HT1 buffer, yielding a 20 pM pooled, denatured library. PhiX (20 pM) was thawed and 30 μL was transferred to a fresh tube, and 570 μL of the 20 pM library was added to the PhiX, resulting in 5% PhiX for library diversification, quality control for cluster generation, sequencing, and alignment. This was mixed and heat shocked at 96° C. for 2 minutes and then immediately placed on ice. The PhiX containing library (600 μL) was added to a well of a 300 cycle v2 Miseq reagent cartridge, and the sequencing reaction was initiated. Following the run, .bam files were used for analysis with IGV software. The results are presented in Table 3.

TABLE 3
NGS Analysis of Editing Efficiencies SpCas9
Nickase RNPs and SpCas9 Nuclease RNPs

| Condition | crRNA | Total Reads | Deletions | Insertions | Indels % |
|---|---|---|---|---|---|
| Control | n/a | 76,030 | 159 | 47 | 0.3 |
| SpCas9 | crRNA-a | 64,605 | 626 | 523 | 1.7 |
| nuclease | crRNA-b | 64,712 | 1570 | 50 | 2.4 |
| RNP | crRNA-c | 70,520 | 1368 | 242 | 2.2 |
| | crRNA-d | 68,572 | 171 | 34 | 0.3 |
| SpCas9 | Design #1 | 58,254 | 7,787 | 45 | 11.9 |
| nickase | Design #2 | 87,394 | 3,990 | 39 | 4.4 |
| RNPs | Design #3 | 53,701 | 301 | 2 | 0.6 |

NGS analysis clearly showed successful genome editing on PD-1 with SpCas9 nickase RNPs in primary T cells. Both design #1 and #2 of SpCas9 nickase RNPs showed higher genome editing efficiencies on PD-1 in primary T cells than SpCas9 nuclease RNPs with any single crRNA. In particular, SpCas9 nickase RNPs with design #1 paired crRNAs (crRNA-a+crRNA-b) resulted in 11.9% indels; while, SpCas9 nuclease RNPs with crRNA-a or crRNA-b resulted in only 1.7% or 2.4% indels.

### Example 3. Evaluation of CRISPR Nickase RNPs on More Immune-Related Targets in K562 Cells

Cytotoxic T-lymphocyte protein 4 (CTLA4), T-cell immunoglobulin and mucin-domain containing-3 (TIM-3; also called Hepatitis A virus cellular receptor 2, HAVCR2) and T-cell receptor alpha constant (TRAC) are emerging targets or genome loci in the cancer immunotherapy landscape. Sets of paired gRNAs were designed for CRISPR-nickase RNPs on these targets (Table 4). The chemically modified single gRNAs (mod-sgRNAs, containing stabilizing 2′-O-methyl and phosphorothioate linkages) were used. The paired mod-sgRNAs were configured in the PAM-out orientation.

TABLE 4
Design of Paired mod-sgRNAs
Design of Paired mod-sgRNAs

| | | | SEQ |
|---|---|---|---|
| | Mod- | | ID |
| Design | sgRNA | sequence | NO: |
| CTLA4 | CTLA4- | UUUGAACCCACACAGAAUCAGUUUUAGAG | 39 |
| pair #1 | gRNA-a | CUAGAAAUAGCAAGUUAAAAUAAGGCUAG | |
| | | UCCGUUAUCAACUUGAAAAAGUGGCACCG | |
| | | AGUCGGUGCUUUUU | |
| | CTLA4- | CCUUGGAUUUCAGCGGCACAGUUUUAGA | 40 |
| | gRNA-b | GCUAGAAAUAGCAAGUUAAAAUAAGGCUA | |
| | | GUCCGUUAUCAACUUGAAAAAGUGGCACC | |
| | | GAGUCGGUGCUUUUU | |
| CTLA4 | CTLA4- | GGAGCGGUGUUCAGGUCUUCGUUUUAGA | 41 |
| pair #2 | gRNA-c | GCUAGAAAUAGCAAGUUAAAAUAAGGCUA | |
| | | GUCCGUUAUCAACUUGAAAAAGUGGCACC | |
| | | GAGUCGGUGCUUUUU | |
| | CTLA4- | GCACAAGGCUCAGCUGAACCGUUUUAGA | 42 |
| | gRNA-d | GCUAGAAAUAGCAAGUUAAAAUAAGGCUA | |
| | | GUCCGUUAUCAACUUGAAAAAGUGGCACC | |
| | | GAGUCGGUGCUUUUU | |
| CTLA4 | CTLA4- | CCUUGUGCCGCUGAAAUCCAGUUUUAGA | 43 |
| pair #3 | gRNA-e | GCUAGAAAUAGCAAGUUAAAAUAAGGCUA | |
| | | GUCCGUUAUCAACUUGAAAAAGUGGCACC | |
| | | GAGUCGGUGCUUUUU | |
| | CTLA4- | GCAAAGGUGAGUGAGACUUUGUUUUAGA | 44 |
| | gRNA-f | GCUAGAAAUAGCAAGUUAAAAUAAGGCUA | |
| | | GUCCGUUAUCAACUUGAAAAAGUGGCACC | |
| | | GAGUCGGUGCUUUUU | |
| TIM-3 | TIM3- | GGCGGCUGGGGUGUAGAAGCGUUUUAGA | 45 |
| pair #1 | gRNA-a | GCUAGAAAUAGCAAGUUAAAAUAAGGCUA | |
| | | GUCCGUUAUCAACUUGAAAAAGUGGCACC | |
| | | GAGUCGGUGCUUUUU | |
| | TIM3- | UGGUGCUCAGGACUGAUGAAGUUUUAGA | 46 |
| | gRNA-b | GCUAGAAAUAGCAAGUUAAAAUAAGGCUA | |
| | | GUCCGUUAUCAACUUGAAAAAGUGGCACC | |
| | | GAGUCGGUGCUUUUU | |
| TIM-3 | TIM3- | UGCCCCAGCAGACGGGCACGGUUUUAGA | 47 |
| pair #2 | gRNA-c | GCUAGAAAUAGCAAGUUAAAAUAAGGCUA | |
| | | GUCCGUUAUCAACUUGAAAAAGUGGCACC | |
| | | GAGUCGGUGCUUUUU | |
| | TIM3- | UGGUGCUCAGGACUGAUGAAGUUUUAGA | 48 |
| | gRNA-d | GCUAGAAAUAGCAAGUUAAAAUAAGGCUA | |
| | | GUCCGUUAUCAACUUGAAAAAGUGGCACC | |
| | | GAGUCGGUGCUUUUU | |
| TIM-3 | TIM3- | ACGUUGCCACAUUCAAACACGUUUUAGAG | 49 |
| pair #3 | gRNA-e | CUAGAAAUAGCAAGUUAAAAUAAGGCUAG | |
| | | UCCGUUAUCAACUUGAAAAAGUGGCACCG | |
| | | AGUCGGUGCUUUUU | |
| | TIM3- | CUAAAUGGGGAUUUCCGCAAGUUUUAGA | 50 |
| | gRNA-f | GCUAGAAAUAGCAAGUUAAAAUAAGGCUA | |
| | | GUCCGUUAUCAACUUGAAAAAGUGGCACC | |
| | | GAGUCGGUGCUUUUU | |
| TRAC | TRAC- | CAGGGUUCUGGAUAUCUGUGUUUUAGAG | 51 |
| pair #1 | gRNA-a | CUAGAAAUAGCAAGUUAAAAUAAGGCUAG | |
| | | UCCGUUAUCAACUUGAAAAAGUGGCACCG | |
| | | AGUCGGUGCUUUUU | |
| | TRAC- | AACAAAUGUGUCACAAAGUAGUUUUAGAG | 52 |
| | gRNA-b | CUAGAAAUAGCAAGUUAAAAUAAGGCUAG | |
| | | UCCGUUAUCAACUUGAAAAAGUGGCACCG | |
| | | AGUCGGUGCUUUUU | |
| TRAC | TRAC- | AGAGUCUCUCAGCUGGUACAGUUUUAGA | 53 |
| pair #2 | gRNA-c | GCUAGAAAUAGCAAGUUAAAAUAAGGCUA | |
| | | GUCCGUUAUCAACUUGAAAAAGUGGCACC | |
| | | GAGUCGGUGCUUUUU | |
| | TRAC- | ACAAAACUGUGCUAGACAUGGUUUUAGAG | 54 |
| | gRNA-d | CUAGAAAUAGCAAGUUAAAAUAAGGCUAG | |
| | | UCCGUUAUCAACUUGAAAAAGUGGCACCG | |
| | | AGUCGGUGCUUUUU | |
| TRAC | TRAC- | GAGAAUCAAAAUCGGUGAAUGUUUUAGAG | 55 |
| pair #3 | gRNA-e | CUAGAAAUAGCAAGUUAAAAUAAGGCUAG | |
| | | UCCGUUAUCAACUUGAAAAAGUGGCACCG | |
| | | AGUCGGUGCUUUUU | |
| | TRAC- | CUUCAAGAGCAACAGUGCUGGUUUUAGA | 56 |
| | gRNA-f | GCUAGAAAUAGCAAGUUAAAAUAAGGCUA | |
| | | GUCCGUUAUCAACUUGAAAAAGUGGCACC | |
| | | GAGUCGGUGCUUUUU | |

SpCas9 nickase RNPs were prepared and delivered into K562 cells as described in Example 1, except that RNPs were assembled at a molar ratio of 3:1 (mod-sgRNA:Cas9 protein).

Genomic DNA was extracted from the K562 cells using a DNA Extraction Solution (Epicentre), and the target sites were PCR amplified (CTLA-4 primers: Forward CTLA-4 primer: 5′-CCCTTGTACTCCAGGAAATTCTCCA, SEQ ID NO: 57, Reverse CTLA-4 primer: 5′-ACTTGTGAGCTCATCCTGAAACCCA, SEQ ID NO: 58; TIM-3 primers: Forward TIM-3 primer: 5′-TCATCCTCCAAACAGGACTGC, SEQ ID NO: 59, Reverse TIM-3 primer: 5′-TGTCCACTCACCTGGTTTGAT, SEQ ID NO: 60; TRAC primers: Forward TRAC primer: 5′-TCAGGTTTCCTTGAGTGGCAG, SEQ ID NO: 61, Reverse TRAC primer: 5′-TGGCAATGGATAAGGCCGAG, SEQ ID NO: 62).

The editing efficiencies of SpCas9 nuclease RNPs were measured by using TIDE/ICE (Tracking of Indels by Decomposition/Inference of CRISPR Edits) assay. Sanger traces were generated by GENEWIZ with target-specific PCR products and analyzed with the TIDE or ICE webtool (http://tide.nki.nl or https://ice.synthego.com). Default parameters were used. Table 5 presents the results.

TABLE 5
Genome editing on CTLA-4, TIM-3 and TRAC
in K562 cells with Dual Cas9 Nickase RNPs

| Condition | | Indel % |
|---|---|---|
| CTLA-4 | SpCas9 nickase + CTLA4 mod-sgRNA pair #1 | 0 |
| | SpCas9 nickase + CTLA4 mod-sgRNA pair #2 | 14 |
| | SpCas9 nickase + CTLA4 mod-sgRNA pair #3 | 3 |
| TIM-3 | SpCas9 nickase + TIM-3 mod-sgRNA pair #1 | 3 |
| | SpCas9 nickase + TIM-3 mod-sgRNA pair #2 | 3 |
| | SpCas9 nickase + TIM-3 mod-sgRNA pair #3 | 18 |
| TRAC | SpCas9 nickase + TRAC mod-sgRNA pair #1 | 3 |
| | SpCas9 nickase + TRAC mod-sgRNA pair #2 | 6 |
| | SpCas9 nickase + TRAC mod-sgRNA pair #3 | 3 |
| Control | | 0 |

As shown in Table 5, successful genome editing on CTLA-4, TIM-3 and TRAC was generated with SpCas9 nickase RNPs in K562 cells. For example, SpCas9 nickase RNPs with CTLA-4 pair #2 resulted in 14% indels; SpCas9 nickase RNPs with TIM-3 pair #3 resulted in 18% indels; and SpCas9 nickase RNPs with TRAC pair #2 resulted in 6% indels, respectively.

### Example 4. Evaluation of CRISPR Nickase RNPs on CTLA-4, TIM-3 and TRAC in Human Primary T Cells

SpCas9 nickase RNPs with highest editing efficiencies on each target in K562 cells (CTLA-4 pair #2, TIM-3 pair #3 and TRAC pair #2) were selected for testing in human primary T cells. SpCas9 nuclease RNPs and SpCas9 nickase RNPs were prepared as described in Example 3; RNPs were delivered into human primary T cells as described in Example 2. The editing efficiencies of SpCas9 nickase RNPs and SpCas9 nuclease RNPs were measured by using TIDE/ICE assay as described in Example 3. The results are presented in Table 6.

Table 6. Genome editing on CTLA-4, TIM-3 and TRAC in Human Primary T Cells with Dual SpCas9 Nickase RNPs and SpCas9 Nuclease RNPs

TABLE 6
Genome editing on CTLA-4, TIM-3 and TRAC
in Human Primary T Cells with Dual SpCas9
Nickase RNPs and SpCas9 Nuclease RNPs

| Condition | | Indel % |
|---|---|---|
| CTLA-4 | SpCas9 nickase + CTLA4 mod-sgRNA pair #2 | 29 |
| | SpCas9 + CTLA4 mod-sgRNA-d | 35 |
| TIM-3 | SpCas9 nickase + TIM-3 mod-sgRNA pair #3 | 11 |
| | SpCas9 + TIM-3 mod-sgRNA-f | 4 |
| TRAC | SpCas9 nickase + TRAC mod-sgRNA pair #2 | 14 |
| | SpCas9 + TRAC mod-sgRNA-d | 36 |
| PD-1 | SpCas9 nickase + PD-1 mod-sgRNA pair #2 | 34 |
| Control | | 0 |

As shown in Table 6, successful genome editing with SpCas9 nickase RNPs on all targets was generated in in human primary T cells. On one of targets, TIM-3, nickase RNPs showed higher genome editing efficiencies in primary T cells than SpCas9 nuclease RNPs. Notably, SpCas9 nickase RNPs with chemical modified single gRNAs on PD-1 (pair #2) resulted in 34% indels in primary T cells, significantly higher than those from nickase RNPs with two parts of cr/tracrRNA (in Example 2, nickase RNPs with PD-1 cr/tracrRNA pairs #2 only resulted in less than 5% indels).

### Example 5. Integration of Donor Polynucleotides Using Paired CRISPR Nickase Ribonucleoproteins (RNPs)

The ability of paired CRISPR nickase RNPs to improve both specificity and the frequency of targeted chromosomal double stranded breaks in eukaryotic cells would also be advantageous for increasing the frequency of integration of exogenous donor polynucleotides. The ability to genetically modify human somatic immune cell genomes with exogenous donor polynucleotides creates many new options to improve immune responses to various diseases (cancer, infectious disease, among others).

Exogenous donor polynucleotides could be used with paired CRISPR nickase RNPs to deliver transgenes to safe harbor loci within eukaryotic immune cells such as the AAVS1 locus (within human gene PPP1R12C), the human Rosa26 locus, Hipp11(H11) locus, or CCR5. Safe harbor loci are defined as location where insertion and expression of exogenous transgenes has minimal impact on the function and health of the cell.

What is claimed is:

1. A method for modifying an immune-related genomic locus in a eukaryotic cell, the method comprising introducing into the eukaryotic cell Clustered Regularly Interspaced Short Palindromic Repeats (CRISPR) nickase ribonucleoproteins (RNPs) comprising a pair of guide RNAs designed to hybridize with target sequences in the immune-related genomic locus, such that repair of a double-stranded break created by the CRISPR nickase RNPs results in modification of the immune-related genomic locus.

2. The method of claim 1, wherein the target sequences of the pair of guide RNAs are on opposite strands of the immune-related genomic locus.

3. The method of claim 1, wherein the pair of guide RNAs is configured such that each protospacer adjacent motif (PAM) sequence adjacent to one of the target sequences is facing outwards (or is located at a distal end of the target sequences).

4. The method of claim 3, where the distance between the PAM sequences is from about 35 base pairs to about 120 base pairs.

5. The method of claim 1, wherein the CRISPR nickase RNP comprises a Cas9 nickase, a Cpf1 nickase, or a Cas13a nickase.

6. The method of claim 5, wherein the CRISPR nickase RNP comprises a Cas9 nickase.

7. The method of claim 6, wherein the Cas9 nickase is a SpCas9 nickase, a FnCas9 nickase, or a SaCas9 nickase.

8. The method of claim 7, wherein the Cas9 nickase is a Cas9-D10A nickase or a Cas9-H840A nickase.

9. The method of claim 1, wherein the CRISPR nickase comprises at least one nuclear localization signal, at least one cell-penetrating domain, at least one marker domain, at least one chromatin disrupting domain, or a combination thereof.

10. The method of claim 1, wherein the molar ratio of the pair of guide RNAs to CRISPR nickase is 0.5:1, 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1.

11. The method of claim 1, wherein the eukaryotic cell is a human cell or a non-human mammalian cell.

12. The method of claim 11, wherein the eukaryotic cell is a primary T cell.

13. The method of claim 1, wherein the pair of guide RNAs is chosen from (a) a guide RNA comprising SEQ ID NO:31 and a guide RNA comprising SEQ ID NO:32, (b) a guide RNA comprising SEQ ID NO:33 and a guide RNA comprising SEQ ID NO:34, (c) a guide RNA comprising SEQ ID NO:33 and a guide RNA comprising SEQ ID NO:32, (d) a guide RNA comprising SEQ ID NO:39 and a guide RNA comprising SEQ ID NO:40, (e) a guide RNA comprising SEQ ID NO:41 and a guide RNA comprising SEQ ID NO:42, (f) a guide RNA comprising SEQ ID NO:43 and a guide RNA comprising SEQ ID NO:44, (g) a guide RNA comprising SEQ ID NO:45 and a guide RNA comprising SEQ ID NO:46, (h) a guide RNA comprising SEQ ID NO:47 and a guide RNA comprising SEQ ID NO:48, (i) a guide RNA comprising SEQ ID NO:49 and a guide RNA comprising SEQ ID NO:50, (j) a guide RNA comprising SEQ ID NO:51 and a guide RNA comprising SEQ ID NO:52, (k) a guide RNA comprising SEQ ID NO:53 and a guide RNA comprising SEQ ID NO:54, or (l) a guide RNA comprising SEQ ID NO:55 and a guide RNA comprising SEQ ID NO:56.

14. The method of claim 1, wherein repair of the double-stranded break by nonhomologous end joining (NHEJ) results in an insertion of at least one nucleotide, a deletion of at least one nucleotide, or a combination thereof, resulting in inactivation of the immune-related genomic locus.

15. The method of claim 1, wherein the method further comprises introducing into the eukaryotic cell a donor polynucleotide comprising a donor sequence having at least one nucleotide change relative to the immune-related genomic locus, and repair of the double-stranded break by homology-directed repair (HDR) results in integration or exchange of the donor sequence into the immune-related genomic locus, resulting in modification of the immune-related genomic locus.

16. The method of claim 1, wherein the immune-related genomic locus is selected from 2B4 (CD244), 4-1BB (CD137), A2aR, AAVS1, ACTB, ALB, B2M, B7.1, B7.2, B7-H2, B7-H3, B7-H4, B7-H6, BAFFR, BCL11A, BLAME (SLAMF8), BTLA, butyrophilins, CCR5, CD100 (SEMA4D), CD103, CD11a, CD11b, CD11c, CD11d, CD150, IPO-3), CD160, CD160 (BY55), CD18, CD19, CD2, CD27, CD28, CD29, CD30, CD4, CD40, CD47, CD48, CD49a, CD49D, CD49f, CD52, CD69, CD7, CD83, CD84, CD8alpha, CD8beta, CD96 (Tactile), CDS, CEACAM1, CRTAM, CTLA4, CXCR4, DGK, DGKA, DGKB, DGKD, DGKE, DGKG, DGKI, DGKK, DGKQ, DGKZ, DHFR, DNAM1 (CD226), EP2/4 receptors, adenosine receptors including A2AR, FAS, FASLG, GADS, GITR, GM-CSF, gp49B, HHLA2, HLA-A, HLA-B, HLA-C, HLA-DPA1, HLA-DPB1, HIV-LTR (long terminal repeat), HLA-DQA1, HLA-DQB1, HLA-DRA, HLA-DRB1, HLA-I, HVEM, HVEM, IA4, ICAM-1, ICOS, ICOS, ICOS (CD278), IFN-alpha/beta/gamma, IL-1 beta, IL-12, IL-15, IL-18, IL-23, IL2R beta, IL2R gamma, IL2RA, IL-6, IL7R alpha, ILT-2, ILT-4, ITGA4, ITGA4, ITGA6, ITGAD, ITGAE, ITGAL, ITGAM, ITGAX, ITGB1, ITGB2, ITGB7, KIR family receptors, KLRG1, LAIR-1, LAT, LIGHT, LTBR, Ly9 (CD229), MNK1/2, NKG2C, NKG2D, NKp30, NKp44, NKp46, NKp80 (KLRF1), OX2R, OX40, PAG/Cbp, PD-1, PD-L1, PD-L2, PGE2 receptors, PIR-B, PPP1R12C, PSGL1, PTPN2, RANCE/RANKL, ROSA26, SELPLG (CD162), SIRPalpha (CD47), SLAM (SLAMF1, SLAMF4 (CD244, 2B4), SLAMF5, SLAMF6 (NTB-A, Ly108), SLAMF7, SLP-76, TGFBR2, TIGIT, TIM-1, TIM-3, TIM-4, TMIGD2, TRA, TRAC, TRB, TRD, TRG, TNF, TNF-alpha, TNFR2, TUBA1, VISTA, VLA1, and VLA-6.

17. The method of claim 1, wherein the immune-related genomic locus is selected from Table A:
TABLE A
Target genomic loci

| | | UniProtKB |
|---|---|---|
| | Gene | Identifier |
| Protein | Symbol | (human) |
| Programmed cell death-1 (PD-1) | PD-1 | Q15116 |
| Cluster of differentiation 52 (CD52) | CD52 | Q9UJ81 |
| Cytotoxic T-lymphocyte protein 4 (CTLA4) | CTLA4 | P16410 |
| Lymphocyte-activation protein 3 (LAG3) | LAG3 | P18627 |
| Integrin lymphocyte function-associated | ITGAL | P20701 |
| antigen 1 (LFA1) comprising integrin | ITGB2 | P05107 |
| alpha L chain (ITGAL) and integrin | | |
| beta 2 chain (ITGB2) | | |
| Hepatitis A virus cellular receptor 2 | HAVCR2 | Q8TDQ0 |
| (HAVCR2) (also called T-cell immuno- | | |
| globulin and mucin-domain containing-3, | | |
| TIM-3) | | |
| T-cell receptor alpha constant (TRAC) | TRAC | P01848 |
| T-cell receptor alpha locus (TCR-alpha) | TRA | A0A0C4ZLG8 |
| T-cell receptor beta locus (TCR-beta) | TRB | A0A0C4ZPA0 |

18. The method of claim 17, wherein the immune-related genomic locus is PD-1, CTLA4, TIM-3, or TRAC.

19. A composition comprising a CRISPR nickase and a pair of guide RNAs engineered to target an immune-related genomic locus.

20. The composition of claim 19, wherein the CRISPR nickase is a Cas9 nickase, a Cpf1 nickase, or a Cas13a nickase.

21. The composition of claim 20, wherein the CRISPR nickase is a Cas9 nickase.

22. The composition of claim 21, wherein the Cas9 nickase is a SpCas9 nickase, a FnCas9 nickase, or a SaCas9 nickase.

23. The composition of claim 22, wherein the Cas9 nickase is a Cas9-D10A nickase or a Cas9-H840A nickase.

24. The composition of claim 1, wherein the pair of guide RNAs is chosen from (a) a guide RNA comprising SEQ ID NO:31 and a guide RNA comprising SEQ ID NO:32, (b) a guide RNA comprising SEQ ID NO:33 and a guide RNA comprising SEQ ID NO:34, (c) a guide RNA comprising SEQ ID NO:33 and a guide RNA comprising SEQ ID NO:32, (d) a guide RNA comprising SEQ ID NO:39 and a guide RNA comprising SEQ ID NO:40, (e) a guide RNA comprising SEQ ID NO:41 and a guide RNA comprising SEQ ID NO:42, (f) a guide RNA comprising SEQ ID NO:43 and a guide RNA comprising SEQ ID NO:44, (g) a guide RNA comprising SEQ ID NO:45 and a guide RNA comprising SEQ ID NO:46, (h) a guide RNA comprising SEQ ID NO:47 and a guide RNA comprising SEQ ID NO:48, (i) a guide RNA comprising SEQ ID NO:49 and a guide RNA comprising SEQ ID NO:50, (j) a guide RNA comprising SEQ ID NO:51 and a guide RNA comprising SEQ ID NO:52, (k) a guide RNA comprising SEQ ID NO:53 and a guide RNA comprising SEQ ID NO:54, or (l) a guide RNA comprising SEQ ID NO:55 and a guide RNA comprising SEQ ID NO:56.

25. A method of treating cancer in a subject, the method comprising modifying an immune-related genomic locus in an ex vivo eukaryotic cell in accordance with claim 1 to prepare a modified eukaryotic cell, and delivering to the subject the modified eukaryotic cell.

26. The method of claim 25, wherein the eukaryotic cell is a T cell or a population of T cells.


SEQUENCE LISTING (63 sequences)

SEQ ID NO: 1 - protein, 7 aa, Artificial Sequence
  Description of Artificial Sequence Synthetic peptide
PKKKRKV

SEQ ID NO: 2 - protein, 7 aa, Artificial Sequence
  Description of Artificial Sequence Synthetic peptide
PKKKRRV

SEQ ID NO: 3 - protein, 16 aa, Artificial Sequence
  Description of Artificial Sequence Synthetic peptide
KRPAATKKAGQAKKKK

SEQ ID NO: 4 - protein, 11 aa, Artificial Sequence
  Description of Artificial Sequence Synthetic peptide
YGRKKRRQRRR

SEQ ID NO: 5 - protein, 9 aa, Artificial Sequence
  Description of Artificial Sequence Synthetic peptide
RKKRRQRRR

SEQ ID NO: 6 - protein, 9 aa, Artificial Sequence
  Description of Artificial Sequence Synthetic peptide
PAAKRVKLD

SEQ ID NO: 7 - protein, 11 aa, Artificial Sequence
  Description of Artificial Sequence Synthetic peptide
RQRRNELKRSP

SEQ ID NO: 8 - protein, 8 aa, Artificial Sequence
  Description of Artificial Sequence Synthetic peptide
VSRKRPRP

SEQ ID NO: 9 - protein, 8 aa, Artificial Sequence
  Description of Artificial Sequence Synthetic peptide
PPKKARED

SEQ ID NO: 10 - protein, 8 aa, Artificial Sequence
  Description of Artificial Sequence Synthetic peptide
PQPKKKPL

SEQ ID NO: 11 - protein, 12 aa, Artificial Sequence
  Description of Artificial Sequence Synthetic peptide
SALIKKKKKMAP

SEQ ID NO: 12 - protein, 7 aa, Artificial Sequence
  Description of Artificial Sequence Synthetic peptide
PKQKKRK

SEQ ID NO: 13 - protein, 10 aa, Artificial Sequence
  Description of Artificial Sequence Synthetic peptide
RKLKKKIKKL

SEQ ID NO: 14 - protein, 10 aa, Artificial Sequence
  Description of Artificial Sequence Synthetic peptide
REKKKFLKRR

SEQ ID NO: 15 - protein, 20 aa, Artificial Sequence
  Description of Artificial Sequence Synthetic peptide
KRKGDEVDGVDEVAKKKSKK

SEQ ID NO: 16 - protein, 17 aa, Artificial Sequence
  Description of Artificial Sequence Synthetic peptide
RKCLQAGMNLEARKTKK

SEQ ID NO: 17 - protein, 38 aa, Artificial Sequence
  Description of Artificial Sequence Synthetic polypeptide
NQSSNFGPMKGGNFGGRSSGPYGGGGQYFAKPRNQGGY

SEQ ID NO: 18 - protein, 42 aa, Artificial Sequence
  Description of Artificial Sequence Synthetic polypeptide
RMRIZFKNKGKDTAELRRRRVEVSVELRKAKKDEQILKRRNV

SEQ ID NO: 19 - protein, 20 aa, Artificial Sequence
  Description of Artificial Sequence Synthetic peptide
GRKKRRQRRRPPQPKKKRKV

SEQ ID NO: 20 - protein, 19 aa, Artificial Sequence
  Description of Artificial Sequence Synthetic peptide
PLSSIFSRIGDPPKKKRKV

SEQ ID NO: 21 - protein, 24 aa, Artificial Sequence
  Description of Artificial Sequence Synthetic peptide
GALFLGWLGAAGSTMGAPKKKRKV

SEQ ID NO: 22 - protein, 27 aa, Artificial Sequence
  Description of Artificial Sequence Synthetic peptide
GALFLGFLGAAGSTMGAWSQPKKKRKV

SEQ ID NO: 23 - protein, 21 aa, Artificial Sequence
  Description of Artificial Sequence Synthetic peptide
KETWWETWWTEWSQPKKKRKV

SEQ ID NO: 24 - protein, 11 aa, Artificial Sequence
  Description of Artificial Sequence Synthetic peptide
YARAAARQARA

SEQ ID NO: 25 - protein, 11 aa, Artificial Sequence
  Description of Artificial Sequence Synthetic peptide
THRLPRRRRRR

SEQ ID NO: 26 - protein, 11 aa, Artificial Sequence
  Description of Artificial Sequence Synthetic peptide
GGRRARRRRRR

SEQ ID NO: 27 - protein, 12 aa, Artificial Sequence
  Description of Artificial Sequence Synthetic peptide
RRQRRTSKLMKR

SEQ ID NO: 28 - protein, 27 aa, Artificial Sequence
  Description of Artificial Sequence Synthetic peptide
GWTLNSAGYLLGKINLKALAALAKKIL

SEQ ID NO: 29 - protein, 33 aa, Artificial Sequence
  Description of Artificial Sequence Synthetic polypeptide
KALAWEAKLAKALAKALAKHLAKALAKALKCEA

SEQ ID NO: 30 - protein, 16 aa, Artificial Sequence
  Description of Artificial Sequence Synthetic peptide
RQIKIWFQNRRMKWKK

SEQ ID NO: 31 - DNA, 23 bp, Artificial Sequence
  Description of Artificial Sequence Synthetic oligonucleotide
gcgtgacttccacatgagcgtgg

SEQ ID NO: 32 - DNA, 23 bp, Artificial Sequence
  Description of Artificial Sequence Synthetic oligonucleotide
gcagttgtgtgacacggaagcgg

SEQ ID NO: 33 - DNA, 23 bp, Artificial Sequence
  Description of Artificial Sequence Synthetic oligonucleotide
gacagcggcacctacctctgtgg

SEQ ID NO: 34 - DNA, 23 bp, Artificial Sequence
  Description of Artificial Sequence Synthetic oligonucleotide
gggccctgaccacgctcatgtgg

SEQ ID NO: 35 - DNA, 22 bp, Artificial Sequence
  Description of Artificial Sequence Synthetic primer
ggacaacgccaccttcacctgc

SEQ ID NO: 36 - DNA, 22 bp, Artificial Sequence
  Description of Artificial Sequence Synthetic primer
ctacgaccctggagctcctgat

SEQ ID NO: 37 - DNA, 60 bp, Artificial Sequence
  Description of Artificial Sequence Synthetic oligonucleotide
  modified_base (34)..(39): a, c, t, g, unknown or other
tcgtcggcagcgtcagatgtgtataagagacagnnnnnnggacaacgccaccttcacctg

SEQ ID NO: 38 - DNA, 62 bp, Artificial Sequence
  Description of Artificial Sequence Synthetic oligonucleotide
  modified_base (35)..(40): a, c, t, g, unknown or other
gtctcgtgggctcggagatgtgtataagagacagnnnnnnctacgaccctggagctcctgat

SEQ ID NO: 39 - RNA, 101 nt, Artificial Sequence
  Description of Artificial Sequence Synthetic polynucleotide
uuugaacccacacagaaucaguuuuagagcuagaaauagcaaguuaaaauaaggcuaguccguuaucaacuugaaaaaguggcaccgagucggugcuuuuu

SEQ ID NO: 40 - RNA, 101 nt, Artificial Sequence
  Description of Artificial Sequence Synthetic polynucleotide
ccuuggauuucagcggcacaguuuuagagcuagaaauagcaaguuaaaauaaggcuaguccguuaucaacuugaaaaaguggcaccgagucggugcuuuuu

SEQ ID NO: 41 - RNA, 101 nt, Artificial Sequence
  Description of Artificial Sequence Synthetic polynucleotide
ggagcgguguucaggucuucguuuuagagcuagaaauagcaaguuaaaauaaggcuaguccguuaucaacuugaaaaaguggcaccgagucggugcuuuuu

SEQ ID NO: 42 - RNA, 101 nt, Artificial Sequence
  Description of Artificial Sequence Synthetic polynucleotide
gcacaaggcucagcugaaccguuuuagagcuagaaauagcaaguuaaaauaaggcuaguccguuaucaacuugaaaaaguggcaccgagucggugcuuuuu

SEQ ID NO: 43 - RNA, 101 nt, Artificial Sequence
  Description of Artificial Sequence Synthetic polynucleotide
ccuugugccgcugaaauccaguuuuagagcuagaaauagcaaguuaaaauaaggcuaguccguuaucaacuugaaaaaguggcaccgagucggugcuuuuu

SEQ ID NO: 44 - RNA, 101 nt, Artificial Sequence
  Description of Artificial Sequence Synthetic polynucleotide
gcaaaggugagugagacuuuguuuuagagcuagaaauagcaaguuaaaauaaggcuaguccguuaucaacuugaaaaaguggcaccgagucggugcuuuuu

SEQ ID NO: 45 - RNA, 101 nt, Artificial Sequence
  Description of Artificial Sequence Synthetic polynucleotide
ggcggcugggguguagaagcguuuuagagcuagaaauagcaaguuaaaauaaggcuaguccguuaucaacuugaaaaaguggcaccgagucggugcuuuuu

SEQ ID NO: 46 - RNA, 101 nt, Artificial Sequence
  Description of Artificial Sequence Synthetic polynucleotide
uggugcucaggacugaugaaguuuuagagcuagaaauagcaaguuaaaauaaggcuaguccguuaucaacuugaaaaaguggcaccgagucggugcuuuuu

SEQ ID NO: 47 - RNA, 101 nt, Artificial Sequence
  Description of Artificial Sequence Synthetic polynucleotide
ugccccagcagacgggcacgguuuuagagcuagaaauagcaaguuaaaauaaggcuaguccguuaucaacuugaaaaaguggcaccgagucggugcuuuuu

SEQ ID NO: 48 - RNA, 101 nt, Artificial Sequence; identical to SEQ ID NO: 46
  Description of Artificial Sequence Synthetic polynucleotide

SEQ ID NO: 49 - RNA, 101 nt, Artificial Sequence
  Description of Artificial Sequence Synthetic polynucleotide
acguugccacauucaaacacguuuuagagcuagaaauagcaaguuaaaauaaggcuaguccguuaucaacuugaaaaaguggcaccgagucggugcuuuuu

SEQ ID NO: 50 - RNA, 101 nt, Artificial Sequence
  Description of Artificial Sequence Synthetic polynucleotide
cuaaauggggauuuccgcaaguuuuagagcuagaaauagcaaguuaaaauaaggcuaguccguuaucaacuugaaaaaguggcaccgagucggugcuuuuu

SEQ ID NO: 51 - RNA, 100 nt, Artificial Sequence
  Description of Artificial Sequence Synthetic polynucleotide
caggguucuggauaucuguguuuuagagcuagaaauagcaaguuaaaauaaggcuaguccguuaucaacuugaaaaaguggcaccgagucggugcuuuuu

SEQ ID NO: 52 - RNA, 101 nt, Artificial Sequence
  Description of Artificial Sequence Synthetic polynucleotide
aacaaaugugucacaaaguaguuuuagagcuagaaauagcaaguuaaaauaaggcuaguccguuaucaacuugaaaaaguggcaccgagucggugcuuuuu

SEQ ID NO: 53 - RNA, 101 nt, Artificial Sequence
  Description of Artificial Sequence Synthetic polynucleotide
agagucucucagcugguacaguuuuagagcuagaaauagcaaguuaaaauaaggcuaguccguuaucaacuugaaaaaguggcaccgagucggugcuuuuu

SEQ ID NO: 54 - RNA, 101 nt, Artificial Sequence
  Description of Artificial Sequence Synthetic polynucleotide
acaaaacugugcuagacaugguuuuagagcuagaaauagcaaguuaaaauaaggcuaguccguuaucaacuugaaaaaguggcaccgagucggugcuuuuu

SEQ ID NO: 55 - RNA, 101 nt, Artificial Sequence
  Description of Artificial Sequence Synthetic polynucleotide
gagaaucaaaaucggugaauguuuuagagcuagaaauagcaaguuaaaauaaggcuaguccguuaucaacuugaaaaaguggcaccgagucggugcuuuuu

SEQ ID NO: 56 - RNA, 101 nt, Artificial Sequence
  Description of Artificial Sequence Synthetic polynucleotide
cuucaagagcaacagugcugguuuuagagcuagaaauagcaaguuaaaauaaggcuaguccguuaucaacuugaaaaaguggcaccgagucggugcuuuuu

SEQ ID NO: 57 - DNA, 25 bp, Artificial Sequence
  Description of Artificial Sequence Synthetic primer
cccttgtactccaggaaattctcca

SEQ ID NO: 58 - DNA, 25 bp, Artificial Sequence
  Description of Artificial Sequence Synthetic primer
acttgtgagctcatcctgaaaccca

SEQ ID NO: 59 - DNA, 21 bp, Artificial Sequence
  Description of Artificial Sequence Synthetic primer
tcatcctccaaacaggactgc

SEQ ID NO: 60 - DNA, 21 bp, Artificial Sequence
  Description of Artificial Sequence Synthetic primer
tgtccactcacctggtttgat

SEQ ID NO: 61 - DNA, 21 bp, Artificial Sequence
  Description of Artificial Sequence Synthetic primer
tcaggtttccttgagtggcag

SEQ ID NO: 62 - DNA, 20 bp, Artificial Sequence
  Description of Artificial Sequence Synthetic primer
tggcaatggataaggccgag

SEQ ID NO: 63 - protein, 6 aa, Artificial Sequence
  Description of Artificial Sequence Synthetic 6xHis tag
HHHHHH